(19) United States
(12) Patent Application Publication
(10) Pub. No.: US 2023/0268066 A1
(43) Pub. Date: Aug. 24, 2023

(54) SYSTEM AND METHOD FOR OPTIMIZED AND PERSONALIZED SERVICE CHECK LIST

(71) Applicant: KONINKLIJKE PHILIPS N.V., EINDHOVEN (NL)

(72) Inventors: Meru Adagouda PATIL, BANGALORE (IN); Ravindra PATIL, BANGALORE (IN); Sarif Kumar NAIK, BANGALORE (IN); Vidya RAVI, BANGALORE (IN)

(21) Appl. No.: 18/015,982

(22) PCT Filed: Jul. 8, 2021
(86) PCT No.: PCT/EP2021/068933

§ 371 Date: Jan. 13, 2023

Related U.S. Application Data
(60) Provisional application No. 63/052,484, filed on Jul. 16, 2020.

(51) Int. Cl. G16H 40/40; G06Q 10/0631

(52) U.S. Cl. CPC G16H 40/40; G06Q 10/06316

(57) ABSTRACT

An apparatus (100) for scheduling field service for a fleet of medical imaging devices includes at least one electronic processor (101, 113) programmed to maintain a service records database (140) storing information on instances of service tasks performed on the fleet including identifiers of service completion times for the instances of service tasks; identify one or more service tasks to be performed on a medical imaging device of the fleet based on content of one or more machine and/or service logs of the medical imaging device to be serviced; optimize a service plan (130) for the upcoming field service based at least on the one or more service tasks to be performed during the upcoming field service and service completion times for instances of the one or more service tasks to be performed in the service records database; and display the service plan on a display device (105).

### FIELD

The following relates generally to the servicing and maintenance arts, especially as directed to medical imaging device servicing or the servicing of other complex systems, maintenance history analysis arts, and related arts.

### BACKGROUND

The maintenance of medical imaging systems (e.g., magnetic resonance (MR), positron emission tomography (PET), computed tomography (CT), interventional—X ray, etc.) is preferably proactive, rather than being reactive to unexpected failures. This can partly be realized by preventively replacing system parts that are statistically likely to fail in the near future. Other maintenance tasks can arise due to problems reported by users, such as non-optimal image quality or hardware or software issues that adversely impact workflow.

Manufacturers of medical devices provide certain years of warranty of the device to the end customer. Once the warranty period expires, a majority of customers opt for an annual maintenance contract for these devices, performed by a field service engineer (FSE) (also referred to herein as an SE). The FSE typically relies on set of checklist or job order sheets that define what kind of activity needs to be carried for each component. The checklist usually includes information such as how to perform tests on the device, a comparisons of an observed outcome with given possible outcomes, tolerance values of the parameters so that FSE can observe the parameter value and compare with standard to check if values are in the range of specification or out of the range, and so forth.

Generally, these checklists or job order sheets are conceptualized during a development phase of the device. Once these documents are created, they are not usually changed over the course of time. The FSE is supposed to complete all the activities listed in the checklist/job order. Often, the checklist can include hundreds of possible tasks to be performed. However, hospital staff do not want to have these imaging systems down for long time intervals to complete servicing of each item in the checklist.

Imaging systems are costly, and system downtime causes loss of revenue to hospitals/diagnostic centers. Therefore, hospitals try to avoid long hours of maintenance activity by limiting FSE access to the imaging system, sometimes resulting in multiple occurrences of rescheduling of the maintenance activity, and/or asking the FSE to fix only critical issues which are manifestly affecting the system, or by not calling for maintenance of the system until the imaging system becomes nonfunctional. In addition, once maintenance activity of the device starts, it is difficult for the FSE to predict how long repairs can take to complete. This can result in the imaging system being down longer than predicted to the customer by the FSE, which adversely impacts customer satisfaction.

The following discloses certain improvements to overcome these problems and others.

### SUMMARY

In one aspect, an apparatus for scheduling field service for a fleet of medical imaging devices includes at least one electronic processor programmed to: maintain a service records database storing information on instances of service tasks performed on the fleet including and service completion times for the instances of service tasks; identify one or more service tasks to be performed during upcoming field service on a medical imaging device to be serviced of the fleet based on content of one or more machine and/or service logs of the medical imaging device to be serviced; optimize a service plan for the upcoming field service based at least on the one or more service tasks to be performed during the upcoming field service and service completion times for instances of the one or more service tasks to be performed in the service records database; and display the service plan on a display device.

In another aspect, a service device includes a display device and at least one user input device. At least one electronic processor is programmed to: provide a user interface (UI) allowing a user to enter one or more inputs via the at least one user input device; retrieve a service plan based on the user inputs, the service plan includes one or more services tasks to be performed on an imaging device, the service plan being displayed on the display device via the UI; and receive iteratively updates to the service plan based on one or more of: the priority level of the identified one or more service tasks; and a completion of one or more of the identified service tasks.

In another aspect, a method for scheduling field service for a fleet of medical imaging devices includes: maintaining a service records database storing information on instances of service tasks performed on the fleet including identifiers of FSEs who performed the instances of service tasks and service completion times for the instances of service tasks; identifying one or more service tasks to be performed during upcoming field service on a medical imaging device to be serviced of the fleet based on content of one or more machine and/or service logs of the medical imaging device to be serviced; identifying a group of two or more of the service tasks to be performed during the upcoming field service which can be performed in parallel based on a model of interconnectivity of parts or systems of the medical imaging device to be serviced; optimizing a service plan for the upcoming field service based at least on the one or more service tasks to be performed during the upcoming field service and service completion times for instances of the one or more service tasks to be performed in the service records database; and displaying the service plan on a display device.

One advantage resides in providing improved medical imaging device maintenance including cost savings by improved diagnostics, reducing the amount of unused parts, and reducing the number of service trips by a FSE.

Another advantage resides in a dynamic and personalized checklist presented to a FSE assigned to service a medical imaging device.

Another advantage resides in reducing an amount of time an FSE needs to service a medical imaging device.

Another advantage resides in a more predictable duration of an FSE service time.

Another advantage resides in reducing unplanned downtimes of a medical imaging device.

Another advantage resides in providing a user interface for a FSE to review information relevant to a service call.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for optimizing a service plan for a medical system in accordance with the present disclosure.

FIG. 2 shows modules implemented by the system of FIG. 1.

FIG. 3 shows exemplary flow chart operations of the system of FIG. 1.

FIG. 4 shows an example service plan generated by the system of FIG. 1.

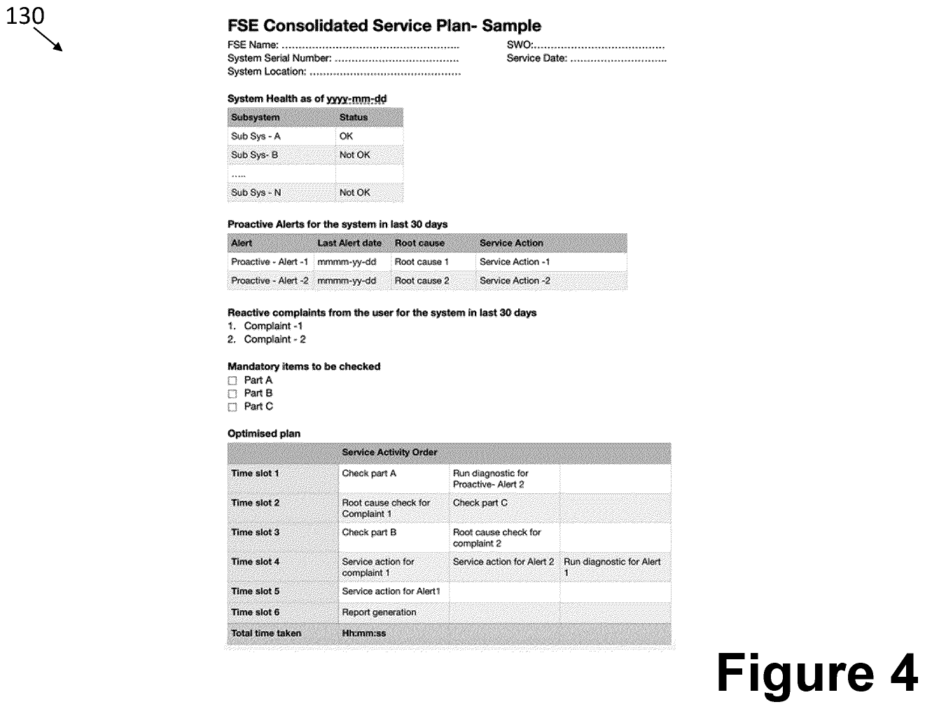

### DETAILED DESCRIPTION

The following relates to a service checklist system for a FSE. At any given time, a range of preventive maintenance and routine servicing tasks can be performed on a complex medical imaging device. A FSE may visit a site to address such preventive/routine servicing tasks. In another scenario, the FSE may be called to diagnose and correct an existing issue that is impairing image quality and/or workflow efficiency. In either case, the FSE ideally would perform all outstanding preventative maintenance and routine servicing tasks during a single visit, in addition to diagnosing and correcting any existing problem. To this end, FSEs currently employ checklists.

However, current FSE checklists are static, and do not account for constraints on the servicing time. In particular, servicing time is often constrained by the customer, for whom servicing downtime translates to lost productivity. In practice, the hospital may only allow the FSE a limited time to work on the imaging device. Furthermore, existing checklists usually do not provide guidance as to how long servicing tasks are likely to take, or which servicing tasks may be more efficiently done together.

The disclosed systems and methods implement a dynamic FSE checklist. For a given imaging device, the system analyzes device historical data (for example, as recorded in the machine log and/or machine service log) and references a table of parts lifespans to identify a list of components that should be serviced (i.e. service tasks).

Additionally, an aggregator collects servicing information from all imaging devices of the fleet, and creates a parts/FSE database of completed service tasks and corresponding service times, indexed as to market, FSE, and other parameters. A similar parts analysis is performed for the list of components using the data in the parts/F SE database in order to estimate service times for the service tasks on the list.

A FSE work analyzer also references the parts/F SE database to identify a ranked list of available FSEs who are best-suited to service the imaging device, based for example on FSE experience with the various service tasks, travel time, and other factors.

A parallelizer analysis is also performed on the list of service tasks for the imaging device, in order to identify any service tasks that can be efficiently done together. For example, if one task requires disassembly of a sub-system then other tasks on the list related to that sub-system may be efficiently performed at the same time.

An optimizer optimizes the service plan based on the foregoing information, together with information obtained from the hospital on time slots available for servicing the imaging device. The optimizer also preferably takes into account a priority ranking of the service tasks, for example prioritizing service tasks deemed to be mandatory either because they are currently manifesting (i.e., the customer has specifically complained about the problem) or because the device vendor has identified certain tasks as mandatory. Optionally, this may be an iterative process.

It will be appreciated that a given implementation of the disclosed dynamic FSE checklist may include subset (rather than all) of the above-listed components or features.

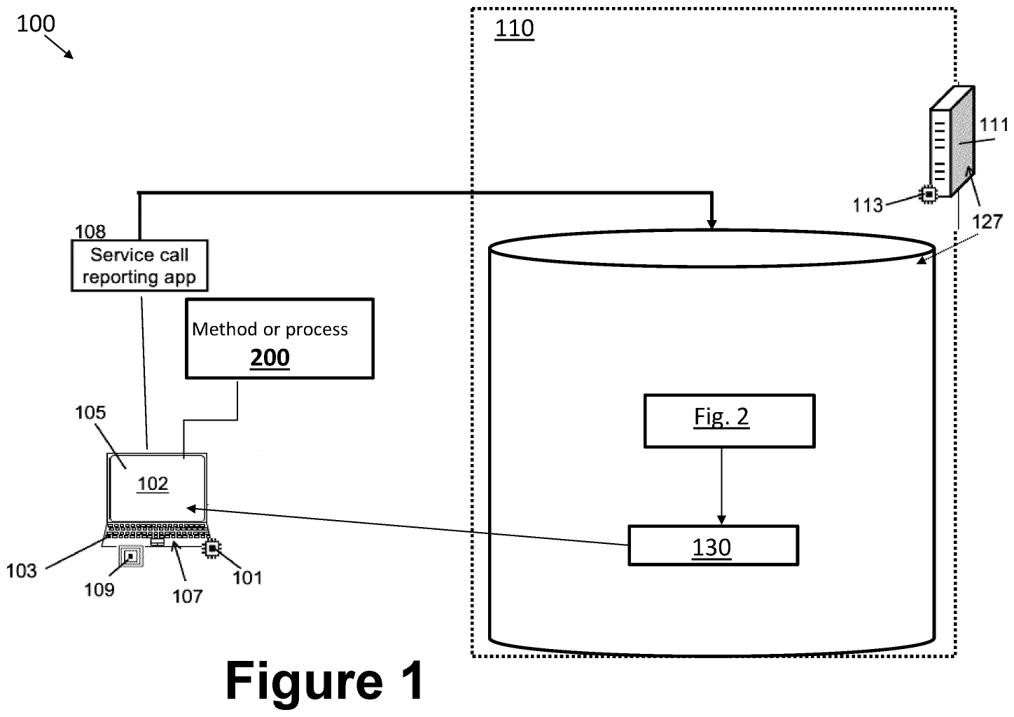

With reference to FIG. 1, an illustrative servicing support system 100 for supporting a service engineer in servicing a device (e.g., a medical imaging device, not shown—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. As shown in FIG. 1, the servicing support system 100 includes, or is accessible by, a service device 102 carried or accessed by a SE. The service device 102 can be a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g. notebook computer, tablet computer, or so forth) carried by an SE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer and the servicing support system 100 may be embodied as an “app” (application program). The service device 102 allows the service engineer to interact with the servicing support system via at least one user input device 103 such a mouse, keyboard or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 to implement the servicing support system 100. The service device 102 may also include a communication interface 109 such that the servicing support system 100 may communicate with a backend server or processing device 111, which may optionally implement some aspects of the servicing support system 100 (e.g., the server 111 may have greater processing power and therefore be preferable for implementing computationally complex aspects of the servicing support system 100). Such communication interfaces 109 include, for example, a wireless Wi-Fi or 4G/5G interface, a wired Ethernet interface, or the like for connection to the Internet and/or an intranet. Some aspects of the servicing support system 100 may also be implemented by cloud processing or other remote processing.

In illustrative FIG. 1, the servicing information collected using a service call reporting app 108 is fed to a database backend 110 (e.g., implemented at a medical facility or other remote center from where the SE is performing the service call, or at the imaging device vendor or other servicing contractor). For example, the database backend 110 may implement a service log for the medical imaging device. The backend processing is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single service device 102, more generally the database backend 110 will receive service call reports from many service devices (e.g., tens, hundreds, or more service devices) carried by different FSEs, and each FSE will be providing a service call report for each service call that the FSE makes (this may total tens or even a few hundred service calls per year by a given FSE). Hence, over time the database backend 110 accumulates a large quantity of service call reporting data.

The non-transitory storage medium 127 stores instructions executable by the electronic processor 113 of the backend server 111 to perform a device service support method or process 200 implemented by the servicing support system 100. In some examples, the method 200 may be performed at least in part by cloud processing. The method 200 result in an output of a service plan or checklist 130 displayed on the display device 105 of the service device 102.

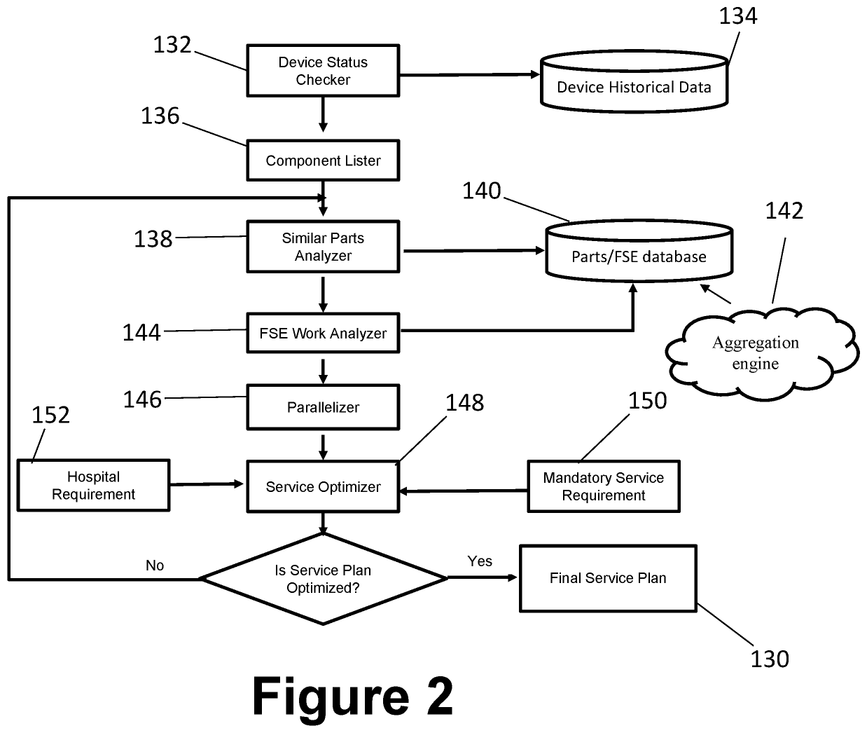

With reference to FIG. 2, and with continuing reference to FIG. 1, the non-transitory computer readable medium 127 stores one or more modules executed by the electronic processor 113 of the backend server 111 (or in some cases the electronic processer 101 of the service device 102) to perform the method 200. As shown in FIG. 2, one of the modules includes a device status checker module 132 configured to analyze a status of a medical imaging device using models and historical service details to provide current health of the medical imaging device. To do so, the device status checker module 132 is configured to continuously monitor the sub-systems of the medical imaging devices, which can be done using a predictive mode and/or a reactive mode. The predictive mode is performed using machine-learning (ML) models that can be trained on historic system data and are modelled to identify drifts in parameter values of the key sensing elements of the subsystem of the medical imaging device. One such example is the pressure of Helium gas for a MRI system. If the model alerting on low helium pressure triggers during or near the planned maintenance window, then this item can be prioritized and appear on the FSE's checklist 130. If the values are within the acceptable limits and the predictive model indicates that this parameter can continue to stay in this acceptable range for the next few months, then this item will be removed from the top of the checklist 130 and can be identified as a low priority item. The priority decision also depends on the presence or absence of system faults and errors in the days just preceding the maintenance activities as well as the day of the activity. These can be categorized as a reactive mode of monitoring. If there are known issues in the medical imaging device, these details are also added to the checklist 130 to provide better decision making.

The device status checker module 132 is in electronic communication with a device historical database 134 that stores historical information about all the medical systems generated from the models. Since the installed base of the medical devices spans multiple geographies, the service records of these systems are aggregated, and data analysis is done on the entire installed base. The crucial elements that are extracted by the device status checker module 132 from the device historical database 134 are the components replaced and associated service actions. The device status checker module 132 is configured to continuously run ML algorithms on this data to identify patterns of component replacements and engineer's comments associated with it to create a mapping between possible causes for replacements. Based on these causes, when this model is running on a system of interest, if there are similar patterns of errors noticed then this required subsystem is also added on to the checklist 130. The device status checker module 132 is configured to continuously map this data into the device historical database 134 by updating root causes and service actions taken around the world so that it can be accessed onsite by any personnel for faster resolution. The device status checker module 132 is configured to identify clusters of causes and replacements that can be used to identify similar errors and replacements.

A component lister module 136 is configured to list one or more components that need to be services based on a current health status of the medical imaging device. The component lister module 136 is configured to use information provided by the device status checker module 132 and the data in the device historical database 134 to identify a list of components that need to be serviced during the FSE visit. The component lister module 136 is also configured to provide a health status based on both the predictive and reactive modes of monitoring from the device status checker module 132. A severity level of the faults is also provided based on the frequency of occurrence of errors and their impact on the medical imaging system performance. If the errors have the potential to lead to a system shut down or lead to unusable system, these issues are indicated as high priority and are placed on the top of the checklist 130. The output of the component lister module 136 is a list of components that need to be checked in the descending order of severity in terms of affecting the system function.

A similar parts analyzer module 138 is configured to compare service action data of the same parts from the historical data and provides most optimal service part along with timing details. For a given imaging system that needs to undergo maintenance, it is necessary to know beforehand the possible issue at site (if any), and the quickest and most efficient way to resolve it. The similar parts analyzer module 138 is configured to analyze the components output from the component lister module 136 and identify if the problem seen at this particular site had appeared in any other site previously from a parts/F SE database 140. In order to identify an actual service action based on historical data and to provide automated service action statements, a language model such as Bidirectional Encoder Representations from Transformers (BERT) can be employed.

The parts/FSE database 140 is configured to store historical data about parts that were replaced, along with a corresponding service path and associated details about the FSE who performed the repair. The parts/FSE database 140 is populated with data from multiple data sources, which are aggregated using an aggregation engine 142 implemented in the backend sever 111. The aggregation engine 142 includes multiple components (not shown in FIG. 2) for formatting and storing the aggregated data in the parts/FSE database 140. For example, the aggregation engine 142 includes a translator module to translate data from the multiple sources in multiple languages into a common language (e.g., English). A data parser is configured to clean the data in order for Natural Language Processing (NLP) operations to be applied to the data. Once the text is normalized, word embedding algorithms (like GloVe or word2vec) are applied in order to find associated word vectors. Training data to train the word embedding models comes from historic service data, and these models are periodically refreshed so that they capture any new fixes that were performed to fix an issue. A data composition module is configured to categorize the data by, for example, identifying common root causes of failure, a diagnosis, service actions and parts replaced to fix the issue, along with a time consumed by the FSE to complete the tasks. This data is categorized to include information such as an identification of the system, a release date, a market where the system is located, the name of the servicing FSE, a symptom of the issue, a diagnosis of the issue, a service action taken, parts to be replace, a time for service, among others, which is stored in the parts/FSE database 140.

A FSE work analyzer 144 is configured to compute an efficiency of an FSE for a given service work, and provide the estimate in terms of time of completion of the work. For example, one of the FSE workers may be experienced in handling a particular failure situation of the part better than the other FSE workers. This might be due to the training, experience and/or familiarity with the prior experience. The FSE work analyzer 144 is configured to assigning the “best” or “most qualified” FSE based on the pertaining problem. To determine the most qualified FSE to handle a particular task or service plan, the FSE work analyzer 144 is configured retrieve, from the parts/FSE database 140, historical data related to about activity time, error rate, dependability, accident rate, turnover time, job experience and knowledge, a distance of FSE from the impacted site, among others (these are merely non-limiting examples). The FSE work analyzer 144 is configured to implement a model (such as a Hidden Markov Model (HMM)) to analyze this retrieved data and output a recommended FSE (and an associated efficiency score) to perform the tasks in the service plan 130.

The following is an example of the HMI (in particular, a second order HMI) implemented by the FSE work analyzer 144. If the set of retrieved data is represented as

X=x₁, x₂, . . . , xₙ and

the activity type and the recommended FSE for the service plan 130 is represented as

Y=y₁, y₂, . . . , yₙ

Based on this data, the HMM is used to define

p(x₁, x₂, . . . , xₙ, y₁, y₂ . . . , yₙ)

for any measurements x₁, x₂, . . . , xₙ & activity type y₁, y₂ . . . , yₙ.
Then the most likely recommended FSE and efficiency for X is represented as

$$arg {max}_{{y}_{1 ,} {y}_{2 ,} , … , {y}_{n ,}} p ( {x}_{1} , {x}_{2} , … , {x}_{n} , {x}_{1} , {y}_{1 ,} {y}_{2 ,} , … . , {y}_{n ,} )$$

$$p ( {x}_{1} , {x}_{2} , … . {x}_{n} , {x}_{1} {y}_{1 ,} {y}_{2 ,} , … . , {y}_{n + 1 ,} ) = {\prod }_{i = 1}^{n + 1} q ( {y}_{i} \vert {y}_{i - 2 ,} {y}_{i - 1} ) {\prod }_{i = 1}^{n} e ( {x}_{i} \vert {y}_{i} )$$

where, q(x|y) and e(x|y) are respectively represent transition and emission probabilities and are estimated using standard maximum likelihood estimation techniques.

Further once the FSE is selected based on the efficiency score, then the time to complete the tasks in the service plan 130 is known.

A parallelizer module 146 is configured to parallelize the service activities (e.g., selecting activities that can be performed parallelly or in tandem). To do so, the parallelizer module 146 is configured to extract detailed steps to resolve the tasks in the service plan 130 from a field service manual (which can be stored in the non-transitory computer readable medium 127) and from a prior field service history data (stored in the parts/FSE database 140) based on a mapping process to the service plan 130. This retrieved data is concatenated by the parallelizer module 146 using NLP processing operations. The concatenated data is identified to determine which are redundant and which can be added to the service plan 130. The parallelizer module 146 is then configured to implement a ML path tracer operation to determine which machine components are connected and which are not. One example of a connected set of components for a task in the service plan 130 includes checking a voltage switch of the medical imaging device by opening a cabinet board, and then charge a battery in the cabinet board while checking the resistance output of the voltage switch. An example of component tasks that are not connected can be a broken display and an X-ray tube calibration, which cannot be performed in tandem. Each of the components are assigned a label as to which other components are interconnected with the selected component.

A service optimizer module 148 is configured to implement mathematical modelling techniques to optimize the service plan 130 based on a pre-requisite mandatory list of tasks 150 and a hospital requirement 152 as need arising from previous stages. The hospital requirement 152 includes requirements from hospital for the tasks in the service plan 130 including a duration for which the device can be relinquished for service. The mandatory service requirement 150 includes a list of service actions to be minimally done to call a given service action as completed. The service optimizer module 148 is configured to analyze the requirements 150 and 152 along with the output of the parallelizer module 146 to optimize the service plan 130.

The following is an example of the operation of the service optimizer module 148. The parallelizer module 146 identifies a list of jobs represented as a list of jobs Jᵢ(Wᵢ,Tᵢ), i=1, 2, . . . , N. where Wᵢ is the work item to be completed with time Tᵢ. The total time available to spare for maintenance is T. The service optimizer module 148 operates as follows: set the mandatory job Jⱼ^{m}(Wⱼ,Tⱼ), j=1, 2, . . . , K. as a task has to be completed in a fixed time i.e.

$${T}^{M} = {\sum }_{j = 1}^{K} {T}_{j} .$$

The total time still available is represented as T^{A}=T−T^{M}.
Among the N jobs Jᵢ, a set of ‘d’ jobs may be the mandatary jobs J^{M}. Hence the remaining jobs are represented as J₁^{A}, where 1=1, 2, . . . , P^{A}, P=N-d. The service optimizer module 148 is then configured to find a set of bets possible jobs O(J₁^{A}(W₁, T₁)) which can be performed in time T^{A}. This is represented as

$${J}_{h}^{S} ( {W}_{h} , {T}_{h} ) , where h = 1 , 2 , … , {P}^{S} , S <= {P}^{A} \\ and \\ {\sum }_{h = 1}^{{P}^{S}} {T}_{h} \leq {T}^{A}$$

Then, the final list of jobs in the service plan 130 is a union of Jⱼ^{M} (Wⱼ, Tⱼ), j=1, 2, . . . , K and Jₕ^{S}(Wₕ, Tₕ)h=1, 2, . . . , P^{S}.

These modules are configured to work in an iterative manner. For example, if the service optimizer module 148 determines that the service plan 130 is not optimized, then the similar parts analyzer 138, FSE work analyzer module 144, and parallelizer module 146 are programmed to repeat their respective operations.

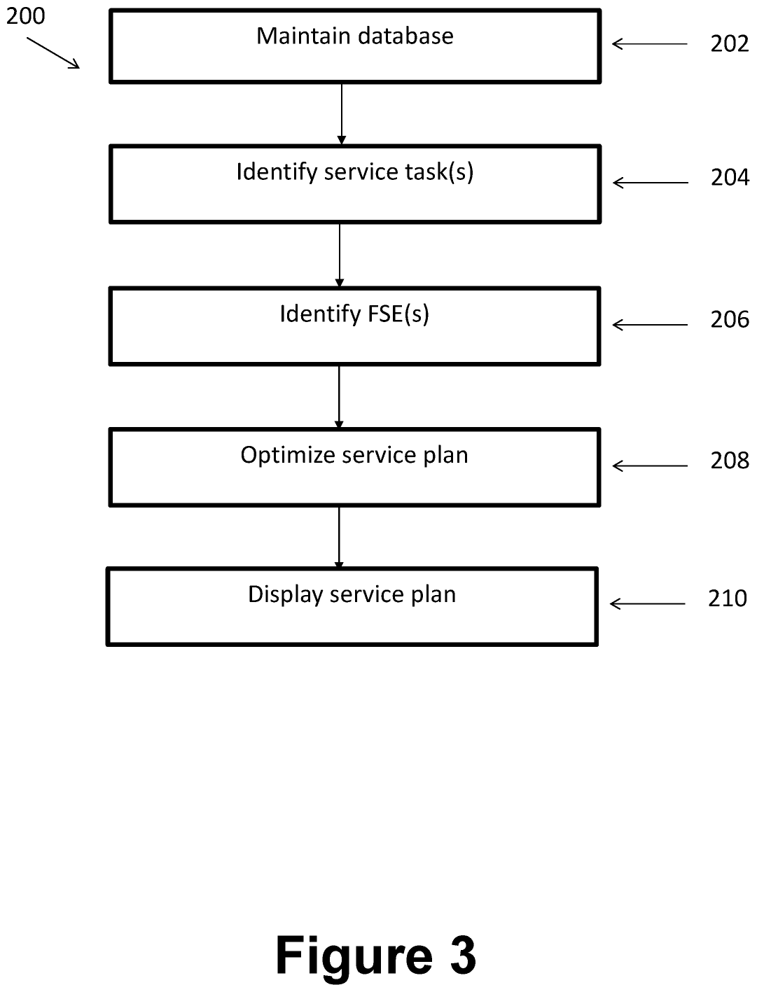

With reference to FIG. 3, and with continuing reference to FIGS. 1 and 2, an illustrative embodiment of an instance of the device service support method 200 executable by the electronic processor 113 is diagrammatically shown as a flowchart.

At an operation 202, a service records database, such as the Parts/FSE database 140 is maintained to store information on instances of service tasks performed on the fleet including identifiers of FSEs who performed the instances of service tasks and service completion times for the instances of service tasks.

At an operation 204, one or more service tasks to be performed during upcoming field service on a medical imaging device to be serviced of the fleet are identified based on content of one or more machine and/or service logs of the medical imaging device to be serviced. The operation 204 can be performed by the similar parts analyzer module 138.

At an operation 206, one or more available FSEs to perform the upcoming field service on the medical imaging device to be serviced are identified (e.g., automatically identified) based on the one or more service tasks to be performed during the upcoming field service and the information stored in the service records database 140. The operation 206 can be performed by the FSE work analyzer module 144.

At an operation 208, a service plan 130 for the upcoming field service is optimized based at least on the one or more service tasks to be performed during the upcoming field service (from the operation 204) and service completion times for instances of the one or more service tasks to be performed in the service records database 140 (from the operation 206).

In some embodiments, the service task identification operation 204 can include ranking the one or more service tasks to be performed during the upcoming field service based on priority levels assigned to the respective service tasks. In this embodiment, the optimization operation 208 can include optimizing the service plan 130 additionally based on the rankings of the service tasks. In some examples, the optimization operation 208 can including iteratively updating the service plan 130 based on, for example, updates to the priority levels of the service tasks, a completion of one or more of the identified services tasks by the FSE, and so forth.

In other embodiments, the service task identification operation 204 includes identify a group of two or more of the service tasks to be performed during the upcoming field service which can be performed in parallel based on a model of interconnectivity of parts or systems of the medical imaging device to be serviced. This process can be performed by the parallelizer module 146. In this embodiment, the optimization operation 208 can include optimizing the service plan 130 additionally based on the identified group of tasks to be performed in parallel.

In some embodiments, the FSE identification operation 206 can include ranking the identified available FSEs based at least on an experience level of the FSEs for the medical imaging device to be serviced and/or the identified one or more service tasks. In this embodiment, the optimization operation 208 can include optimizing the service plan 130 additionally based on the identified one or more available FSEs and the service completion times for instances of the one or more service tasks indicated as performed by the identified one or more FSEs in the Parts/FSE database 140.

In other embodiments, the optimization operation 208 can include optimizing the service plan 130 based on one or more inputs. In one example, a hospital time constraint requirement 152 is received (e.g., from the hospital where the device to be service is disposed) on the upcoming field service, in which the optimization of the service plan is further based on the time constraint. In another example, an indication of a request for one or more service tasks as being mandatory 150 is received, and the optimization of the service plan is further based on the mandatory requirement.

In further embodiments, the maintenance operation 202 can include maintaining the device historical database 134 to include information on parts replaced during the instances of the service tasks. Parts required for the one or more service tasks to be performed during the upcoming field service can be determined based on the information on parts replaced during instances of the one or more service tasks stored in the device historical database 134. The optimization operation 208 includes optimizing the service plan 130 for the upcoming field service is further based on the determined parts required for the one or more service tasks. These processes can be performed by the device status checker module 132 and the components lister module 136.

At an operation 210, the service plan 130 is displayed on the display device 105 of the service device 102. In some examples, the FSE using the service device 102 can retrieve the service plan 130 based on inputs entered via the at least one user input device 103 via a graphical user interface (GUI) displayed on the display device 105. The service plan 130 can be updated on the display device 105 based on, for example, updates to the priority levels in the service tasks in the service plan, completion of one or more the services tasks, and so forth. In other examples, the user inputs can include an indication of a ranking of one or more of the services tasks in the service plan 130 to update the service plan based on how the FSE views the priority of the service tasks. In addition, the user inputs can include inputs to access data in the device historical database 132 and/or the parts/FSE database 140, and update the service plan 130 accordingly.

With reference to FIG. 3, an illustrative example of a service plan 130 is shown. A sample service plan that is generated is customized based on the needs of the system and the customer and mandatory requirements. The service plan 130 indicates the system health by indicating which of the sub-systems are functioning correctly and which are not. It also indicates the proactive alerts generated for the system in a specific time frame (for example, 30 days) before the day of service. The customer complaints that were raised by the users of the system in the specific time frame, (for example, 30 days) is also mentioned. These indicators highlight necessary actions that need to be take along with the regular checklist. There is also a schedule that indicates how the FSE may best cover all the necessary tasks by parallelizing as many tasks as possible.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for scheduling field service for a fleet of medical imaging devices, the apparatus comprising:
at least one electronic processor programmed to:
maintain a service records database storing information on instances of service tasks performed on the fleet including identifiers of service completion times for the instances of service tasks;
identify one or more service tasks to be performed during upcoming field service on a medical imaging device to be serviced of the fleet based on content of one or more machine and/or service logs of the medical imaging device to be serviced;
optimize a service plan for the upcoming field service based at least on the one or more service tasks to be performed during the upcoming field service and service completion times for instances of the one or more service tasks to be performed in the service records database; and
display the service plan on a display device.

2. The apparatus of claim 1, wherein the at least one electronic processor is programmed to:
rank the one or more service tasks to be performed during the upcoming field service based on priority levels assigned to the respective service tasks;
wherein the optimization of the service plan is based at least in part on the ranking.

3. The apparatus of claim 2, wherein the at least one electronic processor is programmed to:
iteratively update the service plan based on one or more of:
updates to the priority levels of the one or more service tasks; and
a completion of one or more of the identified service tasks.

4. The apparatus of claim 1, wherein the at least one electronic processor is programmed to:
identify a group of two or more of the service tasks to be performed during the upcoming field service which can be performed in parallel based on a model of interconnectivity of parts or systems of the medical imaging device to be serviced;
wherein the optimization of the service plan is based at least in part on the identified group.

5. The apparatus of claim 1, wherein the service records database further stores information on field service engineers (FSEs) who performed the instances of service tasks, and the at least one electronic processor is further programmed to:
identify one or more available FSEs to perform the upcoming field service on the medical imaging device to be serviced based on the one or more service tasks to be performed during the upcoming field service and the information stored in the service records database.

6. The apparatus of claim 5, wherein the at least one electronic processor is programmed to:
rank the identified available FSEs based at least on an experience level of the FSEs for the medical imaging device to be serviced and/or the identified one or more service tasks.

7. The apparatus of claim 5, wherein the at least one electronic processor is programmed to optimize the service plan for the imaging device further based on the identified one or more available FSEs and the service completion times for instances of the one or more service tasks indicated as performed by the identified one or more FSEs in the service records data base.

8. The apparatus of claim 1, wherein the at least one electronic processor is programmed to:
receive a time constraint on the upcoming field service;
wherein the optimization of the service plan is further based on the time constraint.

9. The apparatus of claim 2, wherein the at least one electronic processor is programmed to optimize the service plan for the imaging device further based on an indication of a request for one or more service tasks as being mandatory.

10. The apparatus of claim 1, wherein the information on instances of service tasks stored in the service records database further includes information on parts replaced during the instances of the service tasks, and the at least one electronic processor is further programmed to:
determine parts required for the one or more service tasks to be performed during the upcoming field service based on the information on parts replaced during instances of the one or more service tasks stored in the service records database;
wherein the optimization of the service plan for the upcoming field service is further based on the determined parts required for the one or more service tasks.

11. A service device, comprising:
a display device;
at least one user input device; and
at least one electronic processor programmed to:
provide a user interface (UI) allowing a user to enter one or more inputs via the at least one user input device;
retrieve a service plan based on the user inputs, the service plan includes one or more services tasks to be performed on an imaging device, the service plan being displayed on the display device via the UI; and
receive iteratively updates to the service plan based on one or more of:
the priority level of the identified one or more service tasks; and
a completion of one or more of the identified service tasks.

12. The service device of claim 11, wherein the at least one electronic processor is programmed to:
receive one or more user inputs via the at least one user input device including at least a user input indicative of a ranking of one or more of the service tasks in the service plan; and
update the service plan on the UI based on the user inputs.

13. The service device of claim 11, wherein the imaging device comprises a plurality of imaging devices in a fleet of imaging devices, and wherein the at least one electronic processor is programmed to:
receive one or more user inputs via the at least one user input device indicative of a request to retrieve data from a database comprising at least log files, completed service tasks, completed services times, and components information related to the imaging devices in the fleet of imaging devices; and
update the service plan on the UI based on the data retrieved from the database.

14. A method for scheduling field service for a fleet of medical imaging devices, the method comprising:
maintaining a service records database storing information on instances of service tasks performed on the fleet including identifiers of field service engineers (FSEs) who performed the instances of service tasks and service completion times for the instances of service tasks;
identifying one or more service tasks to be performed during upcoming field service on a medical imaging device to be serviced of the fleet based on content of one or more machine and/or service logs of the medical imaging device to be serviced;
identifying a group of two or more of the service tasks to be performed during the upcoming field service which can be performed in parallel based on a model of interconnectivity of parts or systems of the medical imaging device to be serviced;
optimizing a service plan for the upcoming field service based at least on the one or more service tasks to be performed during the upcoming field service and service completion times for instances of the one or more service tasks to be performed in the service records database; and
displaying the service plan on a display device.

15. The method of claim 14, further including:
ranking the one or more service tasks to be performed during the upcoming field service based on priority levels assigned to the respective service tasks;
wherein the optimization of the service plan is based at least in part on the ranking.

16. The method of claim 15, further including:
iteratively update the service plan based on one or more of:
updates to the priority levels of the one or more service tasks; and
a completion of one or more of the identified service tasks.

17. The method of claim 14, further including:
identifying one or more available FSEs to perform the upcoming field service on the medical imaging device to be serviced based on the one or more service tasks to be performed during the upcoming field service and the information stored in the service records database;
wherein the optimization of the service plan is based at least in part on the identified available FSEs.

18. The method of claim 14, further including:
optimizing the service plan for the imaging device further based on an indication of a request for one or more service tasks as being mandatory.

19. The method of claim 14, further including:
optimizing the service plan for the imaging device further based on the identified one or more available FSEs and the service completion times for instances of the one or more service tasks indicated as performed by the identified one or more FSEs in the service records database.

20. The method of claim 14, wherein the information on instances of service tasks stored in the service records database further includes information on parts replaced during the instances of the service tasks, and the method further includes:
determining parts required for the one or more service tasks to be performed during the upcoming field service based on the information on parts replaced during instances of the one or more service tasks stored in the service records database;
wherein the optimization of the service plan for the upcoming field service is further based on the determined parts required for the one or more service tasks.
